(19) United States
(12) Patent Application Publication
(10) Pub. No.: US 2026/0318926 A1
(43) Pub. Date: Oct. 15, 2026

(54) METHOD TO DETERMINE THE EFFICACY OF A NEURODEGENERATIVE DISEASE TREATMENT

(71) Applicant: GRIFOLS WORLDWIDE OPERATIONS LIMITED, DUBLIN (IE)

(72) Inventors: Carla MINGUET PARRAMONA, BARCELONA (ES); Ricardo GONZALO SANZ, BARCELONA (ES); Ana María ORTIZ FERNÁNDEZ, BARCELONA (ES)

(21) Appl. No.: 19/484,797

(22) PCT Filed: May 17, 2024
(86) PCT No.: PCT/EP2024/063729

§ 371 Date: Nov. 14, 2025

(30) Foreign Application Priority Data
May 18, 2023 (EP) 23382467.1

(51) Int. Cl. G01N 33/68; C12Q 1/6883

(52) U.S. Cl. CPC G01N 33/6896; C12Q 1/6883; C12Q 2600/158; G01N 2800/2821; G01N 2800/52

(57) ABSTRACT

The present invention refers to the use of a biomarker for measuring the efficacy or effectiveness of treatments for neurodegenerative diseases, in particular, for Alzheimer's disease.

### FIELD OF THE INVENTION

The present invention refers to the use of a biomarker for measuring the efficacy or effectiveness of treatments for neurodegenerative diseases, in particular, for Alzheimer's disease.

### BACKGROUND OF THE INVENTION

The number of patients suffering from neurodegenerative diseases like Alzheimer's disease (AD) is increasing rapidly along with increasing of old-age population worldwide. Neurodegenerative diseases affect millions of people, greatly reducing their quality of life and, in many cases, causing death.

Alzheimer's disease is a devastating disease of the brain which results in progressive dementia, physical disability and death over a relatively long period of time.

As cognitive dysfunction appears gradually in dementia including AD, there is a disease status of pre-stage of dementia. This stage is called mild cognitive impairment (MCI). MCI is defined as a condition characterized by newly acquired cognitive decline beyond that expected for age or educational background, yet not causing significant functional impairment, and not showing disturbance in daily life. Dementia with Lewy bodies (DLB) is characterized by progressive disease and psychiatric symptoms include anxiety, depression, hallucinations and delusions. The symptoms of DLB are caused by the build-up of Lewy bodies.

Much research is being conducted to develop drugs and new treatments that will slow or halt the progression of these neurodegenerative diseases. However, one of the difficulties in managing these diseases is the lack of means for measuring their progression.

Many techniques have been proposed for measuring the progress of AD. These include cognitive tests which attempt to measure brain functions by having the patient perform different tasks. The problem with this approach is that the ability to measure the progression of the disease using cognitive tests is very limited. Such means are also needed to measure the effectiveness of treatments.

Neurofibrillary tangles (NFTs) and neuritic plaques (NPs) are the classical neuropathological hallmarks of Alzheimer's disease. Numerous neuropathological studies indicate that the first appearance of NFTs and NPs in the hippocampal region of the brain marks the beginning of the degenerative process. Many studies have been done in which the structure of the brain has been imaged to determine structural changes that are linked to the presence and the progression of AD. None have been particularly successful, and in fact, it has been found that profound structural changes can occur in the brain of some individuals with no cognitive impairment or other symptoms of the disease being evident.

In addition to NFTs and NPs, there is growing evidence of a chronic inflammatory response in the AD brain. Such a state of systemic inflammation has been proposed to start or accelerate neurodegenerative processes that eventually result in cognitive decline and AD. Therefore, AD pathogenesis would not be restricted to the neuronal compartment but would involve peripheral inflammatory mechanisms.

Positron emission tomography (PET) and single photon emission computed tomography (SPECT) can be used to monitor regional cerebral glucose metabolism (rCMRglc) and regional cerebral blood flow (rCBF). It has been found that significant hypoperfusion and hypometabolism occur in the region of temporal and parietal association cortices in probable Alzheimer's patients. Many studies have demonstrated the correlation between regional localized hypoperfusion and hypometabolism with cognitive deficits seen on behavioral testing. Despite frequent reports of abnormal function in Alzheimer's patients observed by PET and SPECT, however, the clinical utility of these methods is still controversial.

Functional magnetic resonance imaging (fMRI) technology provides a new approach to study neuronal activity. Conventional fMRI detects changes in cerebral blood volume, flow, and oxygenation that locally occur in association with increased neuronal activity induced by functional paradigms. Much research has been done to find tasks which can be performed by patients, and which reveal in an fMRI image acquired at the same time, regions in the brain that function differently when Alzheimer's disease is present. These efforts have to date been unsuccessful.

Thus, there is a need for new, easy to measure and reliable methods for determining the progression of neurodegenerative diseases including AD in order to evaluate the efficacy or effectiveness of the treatments used.

Biomarkers are a key part of AD research and diagnosis. Biomarkers help researchers and clinicians to detect early brain changes, identify risk factors involved, as well as to categorize participants for clinical trials.

So far, cerebrospinal fluid (CSF) markers (i.e., beta-amyloid protein 42 [Aβ42], the major component of amyloid plaques in the brain, and tau and phospho-tau, the major components of neurofibrillary tangles) have been found to be the closest to utility with the highest diagnostic potential. However, CSF is collected by a lumbar puncture, a procedure that has several convenience drawbacks. Thus, a lumbar puncture cannot be obtained routinely in primary care or in the patient's home, and it does not lend itself to repeated measurement, especially in elderly people such as typical AD patients.

Therefore, identifying sensitive, feasible, and reliable biomarkers for measuring the progression of the disease and the efficacy or effectiveness of treatments for neurodegenerative diseases, in particular, for AD is an unmet medical need.

### SUMMARY OF THE INVENTION

The authors of the present invention have identified a sensitive and reliable biomarker that surprisingly allows to determine whether a specific treatment is being effective in a subject suffering from a neurodegenerative disease, in particular, Alzheimer's disease. Moreover, said biomarker can be detected in a fluid sample of said subject, such as blood, serum or plasma.

Thus, in a first aspect, the present invention refers to an ex vivo method for assessing the effectiveness or efficacy of a treatment in a subject suffering from a neurodegenerative disease, said method comprising:
a) determining the expression level of a MIP1α expression product in a biological sample from said subject;
b) repeating step a) using a biological sample obtained from said subject after a period of treatment; and
c) comparing the expression level in b) with that in a) in order to assess the effectiveness or efficacy of the treatment;
wherein a decrease or a no increase in the expression level in b) compared to a) indicates that the treatment is effective or efficacious in said subject and that said subject is more likely to exhibit a favorable clinical response to said treatment, and wherein an increased expression level in b) compared to a) is indicative of a decreased likelihood of favorable clinical response to said treatment.

In a second aspect, the invention refers to the use of MIP1α as a biomarker of plasma exchange clinical treatment efficacy or effectiveness in a patient suffering from a neurodegenerative disease.

In a further aspect, the invention refers to a kit for predicting whether a patient diagnosed with a neurodegenerative disease, disorder, condition is likely to be responsive or non-responsive to a plasma exchange treatment, or for assessing the efficacy or effectiveness of plasma exchange in treating a patient diagnosed with a neurodegenerative disease, disorder, condition, said kit comprising reagents useful for determining the patients level of MIP1α.

In a further aspect, the invention refers to a method of treating or preventing progression of a neurodegenerative disease in a subject, the method comprising performing a plasma exchange treatment on the subject, wherein a biological sample from the subject has been assayed according to the ex vivo method of the invention to identify that said subject is more likely to exhibit a favorable clinical response to the treatment.

In a further aspect, the invention refers to a method of treating or preventing progression of a neurodegenerative disease in a subject, the method comprising assaying a biological sample from the subject according to the ex vivo method of the invention, and performing a plasma exchange treatment on the subject if the biological sample identifies the subject as being more likely to exhibit a favorable clinical response to the treatment.

In a further aspect, the invention refers to a method of treating or preventing progression of a neurodegenerative disease in a subject, the method comprising performing a plasma exchange treatment on the subject, wherein the subject is identified as more likely to exhibit a favorable clinical response to the treatment based on having, in a biological sample, a decreased level of MIP1α expression level compared to a reference level.

### BRIEF DESCRIPTION OF THE FIGURES

FIG. 1 is a schematic chart of the AMBAR clinical study intervention regimen. Abbreviations: TPE, therapeutic plasma exchange; LVPE, low volume plasma exchange; F, Flebogamma® 5% DIF (intravenous immunoglobulin); A, Albutein® 5%-20% (albumin); S, sham treatment; B, baseline visit; IV, intermediate visit; FV, final visit. The window range for TPE (weeks 1 to 6) is +1 day. For the intermediate visit (weeks 7 to 8), the window range is +2 days. The window range for LVPE (weeks 9 to 53) is +5 days. For the final visit (weeks 54-55), the window range is +2 days.

FIG. 2 is a plot of the effect size changes in inflammatory MIP1α levels with respect to placebo before and after PE-Alb for TPE1 and LVPE7 (acute effects).

FIG. 3 shows predicted trajectories of clinical outcome (change from baseline [CFB] at the end of the study of clinical outcome tests score (ADAS-Cog. CDR-sb, and ADCS-CGIG) according to levels of MIP-1a in placebo and plasma exchange with albumin replacement (PE-Alb)-treated patients. Parsimonious model has been used to generate these plots. Shaded areas represent the 95% confidence interval.

### DETAILED DESCRIPTION OF THE INVENTION

### Definitions

In order to facilitate the understanding of the present description, the meaning of some terms and expressions in the context of the invention will be explained below. Further definitions will be included throughout the description as necessary.

The term “treatment”, as used herein comprises any type of therapy, which aims at terminating, preventing, ameliorating and/or reducing the susceptibility to a clinical condition as described herein. Thus, “treatment,” “treating,” “treat” and the like, as used herein, refer to obtaining a desired pharmacologic and/or physiologic effect, covering any treatment of a pathological condition or disorder in a mammal, including a human. The effect may be prophylactic in terms of completely or partially preventing a disorder or symptom thereof and/or may be therapeutic in terms of a partial or complete cure for a disorder and/or adverse effect attributable to the disorder. That is, “treatment” includes (1) preventing the disorder from occurring or recurring in a subject, (2) inhibiting the disorder, such as arresting its development, (3) stopping or terminating the disorder or at least symptoms associated therewith, so that the host no longer suffers from the disorder or its symptoms, such as causing regression of the disorder or its symptoms, for example, by restoring or repairing a lost, missing or defective function, or stimulating an inefficient process, or (4) relieving, alleviating, or ameliorating the disorder, or symptoms associated therewith, where ameliorating is used in a broad sense to refer to at least a reduction in the magnitude of a parameter.

The terms “clinical responsiveness”, “clinical response” and “treatment response” are used interchangeably and shall be taken to mean a change (improvement or amelioration) of one or more symptoms associated with a disease or disorder, or a therapeutic benefit, which results from the administration of a pharmaceutical composition to a subject in need of said treatment.

The term “responder” shall be taken to mean an individual, subject or patient having a disease or disorder, in particular, a neurodegenerative disease or disorder, wherein a treatment ameliorates or improves one or more symptoms thereof or otherwise provides therapeutic benefit wherein said change results from the administration of a pharmaceutical composition to a subject in need of said treatment.

In the context of treatment, the terms “effectiveness” and “effective” refer to the ability of a treatment to produce an effect under real-world conditions. Similarly, in the context of treatment, the terms “efficacy” and “efficacious” refer to the ability of a treatment to produce an effect under controlled circumstances.

The term “diagnosis”, as used herein, refers both to the process of attempting to determine and/or identify a possible disease in a subject, i.e. the diagnostic procedure, and to the opinion reached by this process, i.e. the diagnostic opinion. As such, it can also be regarded as an attempt at classification of an individual's condition into separate and distinct categories that allow medical decisions about treatment and prognosis to be made. This detection, as it is understood by a person skilled in the art, does not claim to be correct in 100 percent of the analyzed samples. However, it requires that a statistically significant amount of the analyzed samples are classified correctly. The amount that is statistically significant can be established by a person skilled in the art by means of using different statistical tools; illustrative, nonlimiting examples of said statistical tools include determining confidence intervals, determining the p-value, the Student's t-test or Fisher's discriminant functions, etc. The confidence intervals are preferably at least 90 percent, at least 95 percent, at least 97 percent, at least 98 percent or at least 99 percent. The p-value is preferably less than 0.1, less than 0.05, less than 0.01, less than 0.005 or less than 0.0001. The teachings of the present invention preferably allow correctly diagnosing in at least 60 percent, in at least 70 percent, in at least 80 percent, or in at least 90 percent of the subjects of a determined group or population analyzed.

The term “biomarker” refers to any biological entity, preferably mRNA or protein, the occurrence or amount of which is characteristic for a specific situation, for example, a neurodegenerative disease.

The term “correlating” or “correlation”, as used herein, refers to the combination of a biomarker with a clinical parameter for making a prognosis.

The term “macrophage inflammatory protein 1-alpha” (MIP-1-alpha or MIP1α) as used herein, also known as Chemokine (C-C motif) ligand 3 (CCL3), refers to a cytokine belonging to the CC chemokine family that is involved in the acute inflammatory state in the recruitment and activation of polymorphonuclear leukocytes through binding to the receptors CCR1, CCR4 and CCR5. MIP-1α is a major factor produced by macrophages and monocytes that is crucial for immune responses towards infection and inflammation. MIP-1a is also produced by neurons and microglia.

The term “expression level”, as used herein, refers to the measurable quantity of an expression product, produced by a gene, in a sample of the subject, wherein the expression product can be a transcriptional product or a translational product. As understood by the person skilled in the art, the expression level can be quantified by measuring the messenger RNA levels of said gene or by measuring the levels of the protein encoded by said gene. In the context of the present invention, the expression level of the gene encoding MIP1α can be determined by measuring the levels of mRNA encoded by said gene, or by measuring the levels of the protein encoded by said gene, i.e. MIP1α protein or of variants thereof. MIP1α protein variants include all the physiologically relevant post-translational chemical modifications forms of the protein, for example, glycosylation, phosphorylation, acetylation, etc., provided that the functionality of the protein is maintained. Said term encompasses the MIP1α protein of any mammal species, including but not being limited to domestic and farm animals (cows, horses, pigs, sheep, goats, dogs, cats or rodents), primates and humans. Preferably, the MIP1α protein is a human protein.

The term “MIP1α expression product” encompasses RNA (e.g. mRNA), cDNA, peptides or proteins that are encoded by a MIP1α gene. The level (amount or concentration) of any one of these MIP1α expression products within a sample may be referred to as their “expression level” within the sample. Accordingly, the level (amount or concentration) of MIP1α mRNA or cDNA in a sample may be referred to as the expression level of MIP1α mRNA or cDNA in the sample. Similarly, the level (amount or concentration) of MIP1α peptide or protein in a sample may be referred to as the expression level of MIP1α peptide or protein in the sample. Standard methods for determining the level of RNA, mRNA, cDNA, peptides and proteins in a sample are known and may be used in the context of the invention.

As would be clear to a person of skill in the art, it is advantageous to determine the expression level of the same MIP1α expression product in both of steps a) and b) of the method, as this improves the accuracy of the comparison in step c). Accordingly, it is preferred that the MIP1 expression product in step a) and step b) is the same (e.g. MIP1α mRNA in both step a) and b); or MIP1α cDNA in both step a) and b); or MIP1α peptide in both step a) and b); or MIP1α protein in both step a) and b)).

The term “sample” or “biological sample”, as used herein, refers to biological material isolated from a subject. The biological sample contains any biological material suitable for detecting RNA or protein levels. In a particular embodiment, the sample comprises genetic material, e.g., DNA, genomic DNA (gDNA), complementary DNA (cDNA), RNA, heterogeneous nuclear RNA (hnRNA), mRNA, etc., from the subject under study. The sample can be isolated from any suitable tissue or biological fluid such as, for example blood, saliva, plasma, serum, urine, cerebrospinal liquid (CSF), feces, a surgical specimen, a specimen obtained from a biopsy, and a tissue sample embedded in paraffin. Methods for isolating samples are well known to those skilled in the art. In particular, methods for obtaining a sample from a biopsy include gross apportioning of a mass, or micro-dissection or other art-known cell-separation methods. In order to simplify conservation and handling of the samples, these can be formalin-fixed and paraffin-embedded or first frozen and then embedded in a cryosolidifiable medium, such as OCT-Compound, through immersion in a highly cryogenic medium that allows rapid freeze. In a particular embodiment, the sample from the subject according to the methods of the present invention is a biological fluid sample. In a particular embodiment, the sample from the subject according to the methods of the present invention is selected from the group consisting of blood, serum, plasma, and CSF; more preferably from the group consisting of plasma, serum and CSF. Even more preferably, the sample is a serum or a plasma sample. In a particular embodiment, the sample from the subject according to the methods of the present invention is selected from the group consisting of blood, serum, and plasma. In a particular embodiment, the sample is a serum sample.

As would be clear to a person of skill in the art, it is advantageous to determine the use the same type of biological sample in both of steps a) and b) of the method, as this improves the accuracy of the comparison in step c). Accordingly, it is preferred that the biological sample type in step a) and step b) is the same (e.g. blood in both step a) and b); or serum in both step a) and b); or plasma in both step a) and b); or CSF in both step a) and b)).

The term “subject” or “individual” or “animal” or “patient” or “mammal,” relates to all the animals classified as mammals and includes but is not limited to domestic and farm animals, primates and humans, for example, human beings, non-human primates, cows, horses, pigs, sheep, goats, dogs, cats, or rodents. Preferably, the subject is a male or female human being of any age, sex or race.

The terms “cognition”, “cognitive function” and “cognitive performance” are used herein interchangeably and are related to any mental process or state that involves but is not limited to learning, memory, creation of imagery, thinking, awareness, reasoning, spatial ability, speech and language skills, language acquisition and capacity for judgment attention. Cognition is formed in multiple areas of the brain such as hippocampus, cortex and other brain structures.

Cognitive function may be measured by any known method, for example and without limitation, by the clinical global impression of change scale (CIBIC-plus scale); the Mini Mental State Exam (MMSE); the Neuropsychiatric Inventory (NPI); the Clinical Dementia Rating Scale (CDR); the Cambridge Neuropsychological Test Automated Battery (CANTAB) or the Sandoz Clinical Assessment-Geriatric (SCAG). Cognitive function may also be measured indirectly using imaging techniques such as Positron Emission Tomography (PET), functional magnetic resonance imaging (fMRI), Single Photon Emission Computed Tomography (SPECT), or any other imaging technique that allows one to measure brain function.

The term “favorable response” as used herein refers to an improvement in one or more symptoms of a cognitive disorder or condition a patient is affected with. Preferably, it refers to at least a statistically significant improvement of cognitive ability measured as described above. The terms “improving” and “enhancing” may be used interchangeably. For example, according to the present invention, a favorable response of a patient affected by a neurodegenerative disease, such as AD, to treatment may be improvement in the cognitive function, such as improvement in learning, plasticity, and/or long term memory; or reduction in a biomarker such as MIP1α in CSF, blood, plasma or serum.

The term “learning” relates to acquiring or gaining new, or modifying and reinforcing, existing knowledge, behaviors, skills, values, or preferences.

The term “plasticity” relates to synaptic plasticity, brain plasticity or neuroplasticity associated with the ability of the brain to change with learning, and to change the already acquired memory. One measurable parameter reflecting plasticity is memory extinction.

The term “memory” relates to the process in which information is encoded, stored, and retrieved. Memory has three distinguishable categories: sensory memory, short-term memory, and long-term memory.

The term “long term memory” is the ability to keep information for a long or unlimited period of time. Long term memory comprises two major divisions: explicit memory (declarative memory) and implicit memory (non-declarative memory). Long term memory is achieved by memory consolidation which is a category of processes that stabilize a memory trace after its initial acquisition. Consolidation is distinguished into two specific processes, synaptic consolidation, which occurs within the first few hours after learning, and system consolidation, where hippocampus-dependent memories become independent of the hippocampus over a period of weeks to years.

The terms “comprise” and “comprising” are used in the inclusive, open sense, meaning that additional elements may be included.

The term “including” is used herein to mean “including but not limited to” or “including without limitation”. These terms are used interchangeably.

### METHOD OF THE INVENTION

The present invention refers to a biomarker, MIP1α, which can be used to monitor the efficacy or effectiveness of a neurodegenerative disease therapy or treatment. In particular, said MIP1α biomarker can be used for assessing the efficacy or effectiveness of a plasma exchange treatment in a patient suffering from a neurodegenerative disease.

Thus, in a first aspect, the invention refers to an ex vivo method for assessing the effectiveness or efficacy of a treatment in a subject suffering from a neurodegenerative disease, said method comprising:
a) determining the expression level of a MIP1α expression product in a biological sample from said subject;
b) repeating step a) using a biological sample obtained from said subject after a period of treatment; and
c) comparing the expression level in b) with that in a) in order to assess the effectiveness or efficacy of the treatment;
wherein a decrease or a no increase in the expression level in b) compared to a) indicates that the treatment is effective or efficacious in said subject and/or that said subject is more likely to exhibit a favorable clinical response to said treatment, and wherein an increased expression level in b) compared to a) is indicative of a decreased likelihood of favorable clinical response to said treatment.

In the context of this method, the biological sample that is used in step a) is obtained from the subject at an earlier time point than the biological sample that is used in step b) (the latter biological sample being obtained from the subject after a period of treatment). This enables the comparison in step c) to provide information on how the subject is responding to the treatment. The expression level of the MIP1α expression product in the biological sample that is used in step a) can therefore be referred to as a reference (or reference level/reference value/reference sample) herein.

The terms “reference”, “reference level”, “reference sample” and “reference value” are used interchangeably and, as used herein, relate to a predetermined criterion used as a reference for evaluating the values or data obtained from samples collected from a subject. The reference value or reference level can be an absolute value, a relative value, a value that has an upper or a lower limit, a range of values, an average value, a median value or a mean value. The reference level according to the method of the present invention may be obtained from the value of a MIP1α expression product in a biological sample from said subject suffering from a neurodegenerative disease, in particular AD, and which is obtained previous to the treatment referred to in step b) of the method. In one example, the reference level may be obtained from the value of a MIP1α expression product in a biological sample from said subject suffering from a neurodegenerative disease, in particular AD, and which is obtained previous to any treatment for the neurodegenerative disease (in other words, the reference level may be the pre-treatment expression level of the MIP1α expression product in a biological sample from the subject). In this example, step a) comprises determining the pre-treatment expression level of a MIP1α expression product in a biological sample from said subject.

Preferably, said reference level is the level of said biomarker in a reference sample, or a fraction thereof, obtained from the patient before start of treatment wherein the treatment comprises plasma exchange. In a particular case, said reference level is obtained from the value of a MIP1α expression product in a biological sample obtained before start of a therapeutic plasma exchange (TPE) treatment. In another particular case, said reference level is obtained from the value of a MIP1α expression product in a biological sample obtained before start of a low-volume plasma exchange (LVPE) treatment.

In a particular case, said reference level also refers to the level of said biomarker in a sample obtained from a patient before a treatment repetition or iterance when said treatment comprises several repetitions. In other words, said reference level may be the level of said biomarker in a reference sample, or a fraction thereof, obtained from the patient before a treatment repetition wherein the treatment comprises plasma exchange. In a particular case, said reference level is obtained from the value of a MIP1α expression product in a biological sample obtained before start of a therapeutic plasma exchange (TPE) treatment repetition. In another particular case, said reference level is obtained from the value of a MIP1α expression product in a biological sample obtained before start of a low-volume plasma exchange (LVPE) treatment repetition.

The methods described herein comparing the expression level in b) with that in a) in order to assess the effectiveness or efficacy of the treatment, wherein a decrease or no increase in the expression level in b) compared to a) indicates that the treatment is effective or efficacious in said subject and/or that said subject is more likely to exhibit a favorable clinical response to said treatment.

In certain embodiments, the decreased level of MIP1α is lower than the reference by a statistically significant difference. Alternatively, the decreased level of MIP1α means that the concentration is 10 percent, 15 percent, 20 percent, 25 percent, 30 percent, 35 percent, 40 percent, 45 percent, 50 percent, 55 percent, 60 percent, 65 percent, 70 percent, 75 percent, 80 percent, 85 percent, or 90 percent, 95 percent, 100 percent, 3-fold, 4-fold, 5-fold, 6-fold, 7-fold, 8-fold, 9-fold, 10 fold, or lower as compared with the reference concentration.

In certain embodiments, the increased level of MIP1α is increased by a statistically significant difference as compared with the reference. Alternatively, the increased level of MIP1α is increased by 10 percent, 15 percent, 20 percent, 25 percent, 30 percent, 35 percent, 40 percent, 45 percent, 50 percent, 55 percent, 60 percent, 65 percent, 70 percent, 75 percent, 80 percent, 85 percent, 90 percent, 95 percent, 100 percent, 3-fold, 4-fold, 5-fold, 6-fold, 7-fold, 8-fold, 9-fold, 10 fold, or more as compared with the reference concentration.

In a particular embodiment, the sample from the subject according to the method of the invention is a biological fluid sample. In a more particular embodiment, the sample from the subject according to the method of the present invention is selected from the group consisting of blood, serum, plasma, and CSF; more preferably from the group consisting of plasma, serum and CSF. Even more preferably, the sample is a serum or a plasma sample. In a particular embodiment, the sample from the subject according to the method of the present invention is selected from the group consisting of blood, serum, and plasma. In a particular embodiment, the sample is a serum sample.

The term “treatment”, as used herein comprises any type of therapy, which aims at terminating, preventing, ameliorating and/or reducing the susceptibility to a neurodegenerative disease. In a particular embodiment of the invention, said treatment comprises a plasma exchange treatment.

As used herein, the term “plasma exchange” means a procedure in which a patient's blood is passed through a device, for example a plasmapheresis device, and the plasma component filtered or centrifuged by the device is removed and discarded. Red blood cells and other non-filtered blood fractions, optionally along with replacement fluid such as fresh frozen plasma or albumin, are reinfused back into the patient. Traditionally, the efficacy or effectiveness of plasma exchange is proportional to the plasma volume removed in relation to the patient's total plasma volume. In general, a determined volume of plasma is withdrawn from the patient and the plasma exchange is carried out with a volume of a liquid equivalent to approximately 100% of the volume of plasma withdrawn from the patient. A person skilled in the art understands that small variations of approximately +10% of this volume will fall within the scope of the present invention.

Typically, a patient's total blood volume is calculated as per Nadler's formula (Nadler S B, Hidalgo J H, Bloch T. Prediction of blood volume in normal human adults. Surgery. 1962; 51 (2): 224-32), reproduced below:

Patient Total Blood Volume (mL):

Male=(0.006012×H3)/(14.6×W)+604

Female=(0.005835×H3)/(15×W)+183
H=height in inches, W=weight in pounds.

Plasma exchange procedures report the quantity of blood/plasma processed in terms of a patient's total blood volume. Processing 1 Volume equates to processing the patient's total blood volume as determined by Nadler's formula. Similarly, processing 1.5 volumes equates to processing “1.5× the patient's total blood volume” as determined by Nadler's formula. Naturally, volumes over 1 result in some of the patient's blood volume being processed more than once.

Plasma exchanges can also be reported in terms of plasma volumes, e.g. 1 plasma volume. Given that plasma accounts for about 55% of total blood volume the two naming systems are interrelated and ultimately centre upon the total blood volume calculation.

The present invention envisages processing from about 0.25 to about 2 blood volumes by plasma exchange. In some embodiments, from about 0.25 to about 0.5 blood volumes may be subjected to plasma exchange. For example, from about 0.3 to about 0.4 blood volumes may be subjected to plasma exchange. In one embodiment, about 0.33 blood volumes may be subjected to plasma exchange.

In other embodiments, from about 0.75 to about 2 blood volumes may be subjected to plasma exchange. For example, from about 1 to about 2 blood volumes may be subjected to plasma exchange. Such as, from about 1 to about 1.5 blood volumes may be subjected to plasma exchange. In one embodiment, about 1 blood volume may be subjected to plasma exchange.

The skilled person will appreciate that aside from Nadler's formula other less utilised formulae and formulae centred around blood volume averages for a particular range of body weight can also be utilised to determine a patient's total blood volume. Such alternative methodologies are also within the scope of the present invention. For the purposes of the present invention, the relevant variable is blood volume regardless of the method utilised to calculate same. Minor variances in the quantum of blood volume arising from the use of different formulae will not have an effect on the effectiveness or efficacy of the present invention.

In one embodiment, the patient diagnosed with the condition may have from about 10% to about 95% of their plasma removed from their blood as part of the plasma exchange procedure. In some embodiments, from about 10% to about 50% of their plasma may be removed from their blood. In other embodiments, from about 10% to about 40% of their plasma may be removed from their blood. For example, from about 20% to about 40% of their plasma may be removed from their blood. In yet other embodiments, from about 15% to about 30% of their plasma may be removed from their blood.

In further embodiments, from about 50% to about 95% of their plasma may be removed from their blood. In other embodiments, from about 60% to about 95% of their plasma may be removed from their blood. For example, from about 60% to about 90% of their plasma may be removed from their blood. For example, from about 60% to about 85% of their plasma may be removed from their blood. In yet other embodiments, from about 60% to about 80% of their plasma may be removed from their blood. In certain embodiments, from about 60% to about 75% of their plasma may be removed from their blood.

In some embodiments, the patient is subjected to multiple iterances of plasma exchange. For example, each iterance of plasma exchange may occur within 1 to 45 days of the previous iterance. In other embodiments, each iterance of plasma exchange may occur within 1 to 30 days of the previous iterance. For example, each iterance of plasma exchange may occur within 1 to 15 days of the previous iterance. In some embodiments, each iterance of plasma exchange may occur within 1 to 7 days of the previous iterance. In certain embodiments, each iterance of plasma exchange may occur within 1 to 3 days of the previous iterance.

In a particular embodiment of the invention, said plasma exchange refers to therapeutic plasma exchange (TPE). As used herein, the term “therapeutic plasma exchange” or “TPE” refers to a process in which a patient's blood plasma is removed and replaced wherein the removed and replaced plasma volume is approximately that of 1 plasma volume.

In a particular embodiment, such TPE comprises a treatment regimen wherein each iterance of plasma exchange occurs within 1 to 7 days of the previous iterance and wherein the patient is subjected to multiple repetitions of TPE, preferably, the patient is subjected to at least two repetitions of TPE, more preferably to at least three repetitions, even more preferably to at least four repetitions, even more preferably to at least five repetitions, and even more preferably to at least six repetitions.

In another particular embodiment of the invention, said plasma exchange refers to low volume plasma exchange (LVPE).

As used herein, the term “low volume plasma exchange” or “LVPE” refers to a plasma exchange treatment involving low blood volume which is removed and replaced. According to the present invention, low volume of a patient's blood plasma refers to approximately ⅓ plasma volume.

According to a particular embodiment, such LVPE comprises a treatment regimen wherein each iterance of plasma exchange occurs 10-45 days after the previous iterance, more preferably, 15-35 days after the previous itinerance of LVPE, and even more preferably within approximately 30 days of the previous iterance. In a preferred embodiment, the patient is subjected to multiple iterances of LVPE, preferably the patient is subjected to at least two iterances or repetitions of LVPE, more preferably to at least three repetitions, more preferably to at least four repetitions, more preferably to at least five repetitions, more preferably to at least six repetitions, more preferably to at least seven repetitions, more preferably to at least eight repetitions, more preferably to at least nine repetitions, more preferably to at least ten repetitions, more preferably to at least eleven repetitions, and even more preferably to at least twelve repetitions or as many rounds as necessary, even chronically, until the patients shows a positive response.

In one embodiment, said LVPE is carried out with a frequency of one LVPE per month.

In one embodiment, said monthly LVPE is carried out for at least 12 months.

In a particular embodiment, said plasma exchange treatment regimen comprises a combination of therapeutic plasma exchange (TPE) and low volume plasma exchange (LVPE) wherein at least a first iterance of TPE is performed before LVPE. In another particular embodiment, multiple itinerances of conventional therapeutic plasma exchange (TPE) can occur before LVPE. Preferably, the patient is subjected to at least two repetitions of TPE, more preferably to at least three repetitions, even more preferably to at least four repetitions, even more preferably to at least five repetitions, and even more preferably to at least six repetitions before LVPE.

In a preferred embodiment, the patient is subjected to at least six iterances of TPE wherein each iterance of plasma exchange occurs within 5 to 7 days of the previous iterance, followed by at least two repetitions of LVPE, preferably, followed by at least three repetitions of LVPE, more preferably at least four repetitions of LVPE, more preferably, at least five repetitions of LVPE, more preferably, at least six repetitions of LVPE, more preferably, at least seven repetitions of LVPE, more preferably, at least eight repetitions of LVPE, more preferably, at least nine repetitions of LVPE, more preferably, at least ten repetitions of LVPE, more preferably, at least eleven repetitions of LVPE, and even more preferably, followed by at least twelve repetitions of LVPE. In a more preferred embodiment, said plasma exchange treatment comprises a weekly TPE for a period of at least six weeks followed by at least twelve repetitions of LVPE.

In another embodiment, said LVPE treatment is carried out chronically, until whenever the patient shows a positive response.

In order to assess the effectiveness or efficacy of the treatment, the method of the invention comprises: a) determining the expression level of a MIP1α expression product in a biological sample from said subject; and b) repeating step a) using a biological sample obtained from said subject after a period of treatment.

Preferably, said period of treatment is after a plasma exchange treatment. In a more particular embodiment, said period of treatment is after a TPE treatment as explained above and before a LVPE treatment. In another preferred embodiment, the level of a MIP1α expression product can be determined after a TPE treatment followed by a LVPE treatment. In a particular case, the level of a MIP1α expression product can be determined after two or more iterances of LVPE following a TPE treatment. In more particular embodiment, the level of a MIP1α expression product is determined after six or more iterances of LVPE following a TPE treatment, more preferably after twelve or more iterances of LVPE following a TPE treatment.

Depending on the blood volume processed by the plasma exchange procedure (and as a result the percentage of plasma removed from the patient's blood) a patient may receive replacement fluids following a plasma exchange procedure to avoid hypotension and peripheral oedema. Typically, larger blood volumes require the administration of a replacement fluid to compensate for the volumes of plasma removed from the patient's blood. Suitable non-limiting examples of replacement fluids include albumin preparations, and fresh frozen plasma diluted with saline. Smaller blood/plasma volumes, such as those equivalent to plasma donation volumes do not usually require replacement fluids. In a particular embodiment of the invention, said replacement fluid includes an albumin preparation. Thus, in a preferred embodiment of the invention, said plasma exchange treatment, TPE and/or LVPE, includes administering an albumin replacement fluid.

Between 70 and 80% of the oncotic activity in normal human plasma is attributable to its albumin content, which lies in the range of around 35-50 g/L. Consequently, the volume of albumin to be administered as a replacement fluid can be readily calculated based on the blood volume subjected to plasma exchange/the volume of plasma removed from the patient's blood.

As used herein, the term “TPE-Alb” refers to therapeutic plasma exchange (TPE) with albumin replacement. The term “LVPE-Alb” refers to low-volume plasma exchange with albumin replacement.

According to preferred embodiments of the invention, the removed and replaced plasma volume of each TPE is approximately that of 1 plasma volume. Normally, approximately 35 to 45 mL/kg is removed, corresponding to a volume of approximately 2500 mL to 3000 mL for a 70 Kg subject. In a preferred embodiment, the removed plasma volume is replaced with the same volume of replacement solution containing albumin. Accordingly, in certain embodiments the patient may be administered between 10 g and 60 g of albumin per Litre of plasma removed by the TPE procedure. For example, the patient may be administered between 30 g and 55 g of albumin per Litre of plasma removed by the TPE procedure. Preferably, said replacement solution contains approximately between 40 and 50 g of albumin per liter of replaced plasma in the TPE. More preferably, said replacement solution contains approximately 50 g of albumin per liter of replaced plasma in the TPE. This procedure is considered a conventional plasma exchange procedure using standard plasmapheresis device. According to particular embodiments, between 100 and 150 g of albumin can be used for substitution in each round of TPE.

According to preferred embodiments of the invention, the removed and replaced plasma volume of each LVPE is approximately that of ⅓ plasma volume. Normally, the plasma volume removed on each plasmapheresis during the LVPE is between 650 mL and 880 mL. Thus, according to particular embodiments, the patient may be administered between 10 g and 60 g of albumin per Litre of plasma removed by the LVPE procedure. Preferably, the patient may be administered between 20 g and 60 g of albumin per Litre of plasma removed by the LVPE procedure, more preferably, between 20 g and 40 g of albumin per Litre of plasma removed by the LVPE procedure.

The term “albumin” as used herein means a protein having the same and/or very similar tertiary structure as human serum albumin (HSA) or HSA domains and has similar properties of HSA or the relevant domains. The term albumin includes variants, and/or derivatives such as fusions and/or conjugations of an albumin or of an albumin variant. The term “variant” means a polypeptide derived from a parent albumin comprising an alteration, i.e., a substitution, insertion, and/or deletion, at one or more (several) positions. A substitution means a replacement of an amino acid occupying a position with a different amino acid; a deletion means removal of an amino acid occupying a position; and an insertion means adding 1-3 amino acids adjacent to an amino acid occupying a position. The altered polypeptide (variant) can be obtained through human intervention by modification of the polynucleotide sequence encoding the naturally occurring (wild type) albumin.

In a preferred embodiment of the invention the albumin is human albumin, preferably human albumin purified from human plasma.

According to the method of the present invention any treatment that cause a variation, preferably a downregulation, or stabilization in the MIP1α expression level in a subject suffering from a neurodegenerative disease, in particular, Alzheimer's disease, is encompassed within the scope of the present invention. Preferably, said treatment comprises administering albumin or albumin preparations by any administration route. Preferably, albumin is administered by an administration route selected from the group consisting of intravenous, subcutaneous, intramuscular, intradermal, intraperitoneal, intrapulmonary, intranasal, oral, rectal, and combinations thereof.

Also, the albumin can be administered alone or in combination with other compounds, medicaments or compositions used or useful in the treatment of a neurodegenerative disease, in particular, for the treatment of Alzheimer's disease.

As explained above, an iso-oncotic solution of human serum albumin is the most common choice for plasma replacement in a plasma exchange treatment. Naturally, this replacement fluid strategy may lead to a transient decline in the levels of non-albumin plasma constituents including coagulation factors, immunoglobulins, transport proteins, and complement components. In practice, redistribution and re-synthesis keep most other plasma proteins in a satisfactory range. In some circumstances, augmentation therapy with other plasma constituents may be performed should deficiencies arise.

According to a particular embodiment of the invention, said plasma exchange treatment additionally includes administering immunoglobulins.

According to preferred embodiments, said immunoglobulin is IgG. As used herein the term “IgG” is used interchangeably with the term “IVIG”.

In a particular embodiment, during plasma exchange, in particular, during LVPE, albumin infusions as replacement fluids are alternated with infusions of IgG. In preferred embodiments, each IgG round comprises an infusion of about 1 g to about 30 g of IgG, more preferably of about 5 g to about 25 g of IgG, even more preferably of about 10 g to about 20 g of IgG.

In preferred embodiments, at least a first iterance of plasma exchange with albumin replacement is performed before an IgG infusion. More preferably, at least two iterances of plasma exchange with albumin replacement are performed before an IgG infusion. In a preferred embodiment, at least two iterances of TPE with albumin replacement are performed before an IgG infusion round. Preferably, at least two repetitions of TPE with albumin replacement, more preferably at least three repetitions, even more preferably at least four repetitions, even more preferably at least five repetitions, and even more preferably at least six repetitions of TPE with albumin replacement are performed before an IgG infusion round.

Preferably, a first IgG infusion round is performed after several iterances of TPE and before LVPE. More preferably, at least two iterances of LVPE are performed before a new IgG infusion round. More preferably, three iterances of LVPE are performed before a new IgG infusion round.

In order to assess the effectiveness or efficacy of the treatment according to the method of the invention, the method includes a comparison step c) wherein the expression level in b) is compared to the expression level in a) in order to assess the effectiveness or efficacy of the treatment.

According to the method of the invention a decrease or a no increase in the MIP1α expression level indicates that the treatment is effective or efficacious and that said subject is more likely to exhibit a favorable clinical response to said treatment and an increased expression level is indicative of a decreased likelihood of favorable clinical response to said treatment.

According to a particular embodiment, the comparison step (c) is performed by comparing the MIP1α expression level in a) with a single time point in b), i.e. after a period of treatment. Alternatively, said comparison step c) may be made between the MIP1α expression level in a) and a plurality of time points in b) (measured at two or more succeeding time points during the treatment period in order to see the tendency of MIP1α expression level). In other words, the comparison step c) may be between one reference value (from step a) and one or more test values (from step b), where the test values are determined from biological samples that were obtained from the subject after a period of treatment (e.g. from subsequent time points during treatment). More preferably, at two or more subsequent time points during the LVPE treatment period.

In another preferred embodiment, said comparison step c) is made between the MIP1α expression level in a) and a slope calculated from a plurality of measurements of said level in b) measured at two or more succeeding time points during the treatment period. More preferably, at two or more subsequent time points during the LVPE treatment period.

According to the method of the invention, said subject is a mammal, preferably a human.

The method of the invention can be used for determining the effectiveness or efficacy of a treatment in a subject suffering from neurodegenerative disease. In a preferred embodiment, said neurodegenerative disease is selected from the group consisting of Parkinson's disease, synucleinopathies, Alzheimer's disease, Mild Cognitive Impairment, Diffuse Lewy body disease, Dementia with Lewy bodies type, amyotrophic lateral sclerosis, multiple sclerosis, Pick's disease, tauopathies, trinucleotide repeat expansion diseases such as (without limitation) Huntington's disease and spinocerebellar ataxias, Creutzfeldt-Jakob disease, frontotemporal dementia, and combinations thereof. In a particular embodiment, said neurodegenerative disease is selected from the group consisting of Parkinson's disease, synucleinopathies, Alzheimer's disease, Mild Cognitive Impairment, Diffuse Lewy body disease, Dementia with Lewy bodies type, multiple sclerosis, Pick's disease, tauopathies, trinucleotide repeat expansion diseases such as (without limitation) Huntington's disease and spinocerebellar ataxias, Creutzfeldt-Jakob disease, frontotemporal dementia, and combinations thereof. Preferably, said neurogenerative disease is a disease wherein neuroinflammation occurs or wherein neuroinflammation plays a role in the progression of the disease or drives the disease. More preferably, said disease is selected from Alzheimer's disease and Mild Cognitive Impairment. Even more preferably, said neurodegenerative disease is Alzheimer's disease.

The Alzheimer's disease may be mild to moderate. For example, the patient suffering from Alzheimer's disease may have a mini mental state examination (MMSE) score of between 10-26, such as for example 18-26. For example, the Alzheimer's disease patient may have a MMSE score of between 10-15, 15-26, or 18-26.

### MIP1α Expression Level

As explained above, the term “expression level” refers to the measurable quantity of an expression product produced by MIP1α gene in a sample of the subject. As used herein, the term “expression product” refers to a transcriptional product or a translational product. In a particular embodiment, said MIP1α expression product is the expression level of a MIP1α RNA transcript, in particular, the mRNA, or its expression product, i.e. the protein encoded by the MIP1α gene or variants thereof. MIP1α protein variants include all the physiologically relevant post-translational chemical modifications forms of the protein, for example, glycosylation, phosphorylation, acetylation, etc., provided that the functionality of the protein is maintained. Preferably, the MIP1α protein is a human protein.

Suitable methods to determine gene expression levels at the mRNA level include, without limitation, standard assays for determining mRNA expression levels such as qPCR, RT-PCR, RNA protection analysis, Northern blot, RNA dot blot, in situ hybridization, microarray technology, tag based methods such as serial analysis of gene expression (SAGE) including variants such as LongSAGE and SuperSAGE, microarrays, fluorescence in situ hybridization (FISH), including variants such as Flow-FISH, qFISH and double fusion FISH (D-FISH), and the like.

Suitable methods to determine gene expression levels at the protein level include, without limitation, conventional methods for determining protein expression levels, such as using antibodies with a capacity to specifically bind to the proteins encoded by said genes (or to fragments thereof containing antigenic determinants) and subsequent quantification of the resulting antibody-antigen complexes.

In a particular embodiment, MIP1α protein levels can be quantified by using standard assays for determining protein expression levels such as Western-blot or Western transfer, ELISA (enzyme-linked immunosorbent assay), immunoassays such as RIA (radioimmunoassay), competitive EIA (competitive enzyme immunoassay), MSD (Meso Scale Discovery) immunoassays, DAS-ELISA (double antibody sandwich ELISA), immunocytochemical and immunohistochemical techniques, techniques based on the use of protein biochips or microarrays which include specific antibodies or assays based on colloidal precipitation in formats such as dipsticks, flow cytometry, confocal microscopy, enzymatic assays, surface plasmon resonance and PAGE-SDS.

As used herein the term “antibody” is used in a broader sense and includes whole antibodies and any antigen binding fragments or derivatives (i.e., “antigen-binding portion”). It may specifically cover polyclonal sera, hybridoma supernatants or monoclonal antibodies, antibody fragments, Fv, Fab, Fab′ and F(ab′)2, ScFv, diabodies, triabodies, tetrabodies and humanized antibodies. At the same time, the antibodies can be labeled or not. Illustrative, but non-exclusive examples of markers which can be used include radioactive isotopes, enzymes, fluorophores, chemiluminescent reagents, enzymatic substrates or cofactors, enzymatic inhibitors, particles, colorants, etc. There are a wide variety of well-known assays that can be used in the present invention, which use non-labeled antibodies (primary antibody) and labeled antibodies (secondary antibodies); among these techniques are included Western blot or Western transfer, ELISA, RIA, competitive EIA, DAS-ELISA, immunocytochemical and immunohistochemical techniques, techniques based on the use of biochips or protein microarrays including specific antibodies or assays based on colloidal precipitation in formats such as dipsticks. Other ways of detecting and quantifying the levels of the protein of interest include techniques of affinity chromatography, binding-ligand assays, etc.

On the other hand, the determination of the levels of the MIP1α protein can be carried out by constructing a tissue microarray (TMA) containing the subject samples assembled and determining the expression levels of the corresponding protein by immunohistochemistry techniques. The immunostaining intensity can be evaluated by using imaging techniques and automated methods. In another preferred embodiment, the expression level of the expression product is determined by immunohistochemistry. In another particular embodiment, the expression level of the expression product is determined by proteomics technology. These techniques are well known to those skilled in the art, including any minor variations as will be readily apparent.

A naturally occurring “antibody” is a glycoprotein comprising at least two heavy (H) chains and two light (L) chains inter-connected by disulfide bonds. Each heavy chain is comprised of a heavy chain variable region (abbreviated herein as VH) and a heavy chain constant region. The heavy chain constant region is comprised of three domains, CH1, CH2 and CH3. Each light chain is comprised of a light chain variable region (abbreviated herein as VL) and a light chain constant region. The light chain constant region is comprised of one domain, CL. The VH and VL regions can be further subdivided into regions of hypervariability, termed complementarity determining regions (CDR), interspersed with regions that are more conserved, termed framework regions (FR). Each VH and VL is composed of three CDRs and four FRs arranged from amino-terminus to carboxy-terminus in the following order: FR1, CDR1, FR2, CDR2, FR3, CDR3, FR4. The variable regions of the heavy and light chains contain a binding domain that interacts with an antigen.

An “isolated antibody”, as used herein, refers to an antibody that is substantially free of other antibodies having different antigenic specificities. Moreover, an isolated antibody may be substantially free of other cellular material and/or chemicals.

The terms “monoclonal antibody” and “monoclonal antibody composition” as used herein refer to a preparation of antibody molecules of single molecular composition. A monoclonal antibody composition displays a single binding specificity and affinity for a particular epitope.

The term “recombinant human antibody”, as used herein, includes all human antibodies that are prepared, expressed, created or isolated by recombinant means, such as antibodies isolated from an animal (e.g., a mouse) that is transgenic or transchromosomal for human immunoglobulin genes or a hybridoma prepared therefrom, antibodies isolated from a host cell transformed to express the human antibody, e.g., from a transfectoma, antibodies isolated from a recombinant, combinatorial human antibody library, and antibodies prepared, expressed, created or isolated by any other means that involve splicing of all or a portion of a human immunoglobulin gene, sequences to other DNA sequences. Such recombinant human antibodies have variable regions in which the framework and CDR regions are derived from human germline immunoglobulin sequences. In certain embodiments, however, such recombinant human antibodies can be subjected to in vitro mutagenesis (or, when an animal transgenic for human Ig sequences is used, in vivo somatic mutagenesis) and thus the amino acid sequences of the VH and VL regions of the recombinant antibodies are sequences that, while derived from and related to human germline VH and VL sequences, may not naturally exist within the human antibody germline repertoire in vivo.

As used herein, “isotype” refers to the antibody class (e.g., IgM, IgE, IgG such as IgG1 or IgG4) that is provided by the heavy chain constant region genes.

In a further embodiment, the invention also refers to a method for predicting whether a patient diagnosed with a neurodegenerative disease, disorder or condition is likely to be responsive or non-responsive to a plasma exchange treatment, said method comprising determining ex vivo, in a fluid sample obtained from the patient the expression level of a MIP1α expression product, wherein an equal or decreased expression level in the blood sample as compared to a reference level indicates that the patient is likely to be responsive to treatment, and an increased level of said biomarker in the blood sample as compared to said reference indicates that the patient is likely to be non-responsive to treatment, wherein the sample is obtained from the patient after treatment and the level of said biomarker in said sample, or fraction thereof, is compared with a reference, which is the level of said biomarker in a reference sample, or a fraction thereof, obtained from the patient before start of treatment or the level of said biomarker in said sample, or a fraction thereof, of a healthy human population.

In particular embodiments, in case the patient is likely to be responsive, said treatment is continued; and in case the patient is likely to be non-responsive, said treatment is discontinued.

### Uses of the Biomarker of the Invention

Means for measuring the progression of a neurodegenerative disease and the effectiveness or efficacy of treatments for neurodegenerative diseases, in particular, for AD is an unmet medical need and an aspect of the present invention.

Thus, in another aspect, the invention refers to the use of MIP1α as a biomarker of clinical treatment efficacy or effectiveness in a patient suffering from a neurodegenerative disease.

The terms “plasma exchange” and “treatment efficacy” or “neurodegenerative disease” have been defined above and are equally applicable to the uses according to the present invention.

In a preferred embodiment, said clinical treatment comprises plasma exchange. In a preferred embodiment, said neurodegenerative disease is selected from the group consisting of Parkinson's disease, synucleinopathies, Alzheimer's disease, Mild Cognitive Impairment, Diffuse Lewy body disease, Dementia with Lewy bodies type, amyotrophic lateral sclerosis, multiple sclerosis, Pick's disease, tauopathies, trinucleotide repeat expansion diseases such as (without limitation) Huntington's disease and spinocerebellar ataxias, Creutzfeldt-Jakob disease, frontotemporal dementia, and combinations thereof. In a particular embodiment, said neurodegenerative disease is selected from the group consisting of Parkinson's disease, synucleinopathies, Alzheimer's disease, Mild Cognitive Impairment, Diffuse Lewy body disease, Dementia with Lewy bodies type, multiple sclerosis, Pick's disease, tauopathies, trinucleotide repeat expansion diseases such as (without limitation) Huntington's disease and spinocerebellar ataxias, Creutzfeldt-Jakob disease, frontotemporal dementia, and combinations thereof. Preferably, said neurogenerative disease is a disease wherein neuroinflammation occurs or wherein neuroinflammation plays a role in the progression of the disease or drives the disease. More preferably, said disease is selected from Alzheimer's disease and Mild Cognitive Impairment. Even more preferably, said neurodegenerative disease is Alzheimer's disease.

Many techniques have been described for measuring the progress of AD. These include cognitive tests which attempt to measure brain functions by having the patient perform different tasks. The problem with this approach is that the ability to measure the progression of the disease using cognitive tests is very limited.

As shown in the Examples below, the inventors have clearly demonstrated that, in serum, MIP1α significantly correlated (p<0.05) with three neuropsychological tests assessed: ADAS-Cog, CDR-sb, and ADCS-CGIC.

The Alzheimer's Disease Assessment Scale-cognitive test (ADAS-Cog) is specifically designed to evaluate the severity of the fundamental alterations in cognitive and behavioral function that are characteristic of patients with AD. The total ADAS-Cog score (0-80; higher score indicates more cognitive impairment) was analyzed as a continuous variable.

The Clinical Dementia Rating Sum of Boxes (CDR-Sb) assesses 6 different domains of dementia: memory, orientation, judgment and problem solving, community affairs, home and hobbies, and personal care. Each domain (other than personal care) is given a score of 0, 0.5, 1, 2 or 3. The personal care domain is scored as 0, 1, 2 or 3. Higher scores indicate more severe dementia.

The Alzheimer's Disease Cooperative Study-Clinical Global Impression of Change (ADCS-CGIC), is performed by interviewing the patient to assess function and mental status and the informant, using a worksheet that comprehensively lists relevant symptoms potentially useful in judging clinically meaningful change, and allows for notes for future reference. Scoring goes from 1 (marked improvement) to 7 (marked worsening).

As it is shown in the Examples herein, correlation significance results coincided using two statistic models, the parsimonious and the extended MMRM (Mixed Models for Repeated Measures) models. Changes in MIP1α levels significantly correlated with three neuropsychological tests: ADAS-Cog, CDR-sb, and ADCS-CGIC. Thus, it has been clearly demonstrated that there is a clear relationship between the levels of MIP1α biomarker and clinical outcome.

### Kits of the Invention

In still an additional aspect, the present invention provides a kit for assessing the efficacy or effectiveness of plasma exchange in treating a patient diagnosed with a neurodegenerative disease, disorder or condition, said kit comprising reagents useful for determining the patients level of MIP1α. Suitable reagents for binding specifically with a protein biomarker such as MIP1α include, without limitation, antibodies. In a particular embodiment, said kit comprises an antibody, or antigen-binding fragment thereof, that specifically binds to MIP1α.

As used herein, the term “kit” is used in reference to a combination of articles that facilitate the methods of the present invention. These kits provide the materials necessary for carrying out the application described herein.

The kit may comprise, in addition, a packaging which allows maintaining the reagents within determined limits. Suitable materials for preparing such packings include glass, plastic (polyethylene, polypropylene, polycarbonate and the like), bottles, vials, paper, sachets and the like. The kit of the invention can additionally contain instructions for using the components contained therein. Said instructions can be found in the form of printed material or in the form of an electronic support which can store instructions such that they can be read by a subject, such as electronic storage media (magnetic disks, tapes and the like), optical media (CD-ROM, DVD) and the like. The media can additionally or alternatively contain Internet websites providing said instructions.

In a particular embodiment, MIP1α expression levels to be determined in the kit of the invention are determined as MIP1α protein levels. In a more particular embodiment, MIP1α protein levels are determined by immunoassay, ELISA, western blot or by immunohistochemistry.

The invention is described below by the following examples, which must be considered as merely illustrative and in no case limiting of the scope of the present invention.

### EXAMPLES

### Material and Methods

### Study Design

The study was performed on serum samples from patients participating in the AMBAR study (EudraCT #: 2011-001598-25; ClinicalTrials.gov ID: NCT01561053), in which 322 individuals diagnosed with mild to moderate Alzheimer's disease (Mini-Mental State Examination [MMSE] score from 18 to 26) were enrolled [Boada M, et al (2019. Alzheimers Dement 26, 5:61-69].

Patients underwent a 14-month treatment program of plasma exchange with albumin replacement (PE-Alb) for AD treatment.

MIP1α levels immediately before and after PE-Alb as well as changes from baseline to several representative time points across the study were assessed (see sampling below).

### Treatment Groups

In the AMBAR trial, patients were randomized to one of three PE-Alb treatment groups or to a control group (sham PE) in a 1:1:1:1 fashion. The control (placebo) group underwent a simulated PE treatment through a noninvasive procedure (sham) that mimicked PE but without any actual fluid replacement.

The intervention regime lasted 14 months, which included a first baseline visit, a first 6-week stage of intensive treatment with one session of conventional therapeutic plasma exchange (TPE) with replacement albumin (5% Albutein®, Grifols) per week for all the active groups, followed by an intermediate visit and a second 12-month stage of maintenance treatment with one session of low-volume plasma exchange (LVPE) per month with replacement albumin (20% Albutein®, Grifols) and with or without IVIG (Flebogamma® 5% DIF, Grifols) according to three PE-Alb treatment modalities:
(LA) low dose albumin (20 g Albumin per PE procedure),
(LAF) low dose albumin (20 g Albumin per PE procedure) alternated with infusions of IVIG (F) (infusion of IVIG 10 g per PE procedure); and
(HAF) high dose albumin (40 g Albumin per PE procedure) alternated with infusions of IVIG (F) (IVIG 20 g per PE procedure).

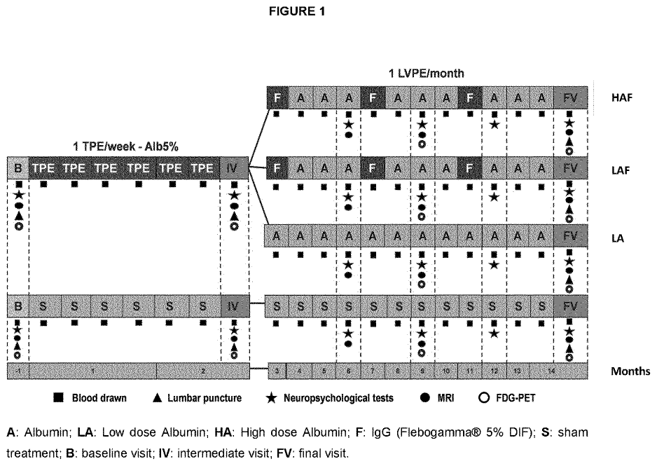

A final follow-up visit at month 14 closed the study. Treatment periods and treatment groups are summarized in FIG. 1.

The removed and replaced plasma volume of each TPE was approximately that of 1 plasma volume and it depended on the patient's sex, height, weight, and hematocrit (approximately 35 to 45 mL/kg, corresponding to a volume of approximately 2500 mL to 3000 mL). This volume was calculated automatically by the device or manually by the operator depending on the device used. The removed plasma volume was replaced with the same volume of albumin 5% during the procedure (50 g/L, approximately 125 g to 150 g albumin). This procedure is a conventional plasma exchange and each site performed the TPE using its standard plasmapheresis device.

Therapeutic plasma exchange is performed using a commercial continuous flow cell separator with either centrifugation- or filtration-based technology. Either a peripheral (e.g., radial/cubital vein) or central access (e.g., subclavian/jugular vein) is used based on the individual characteristics of the patient.

The plasma volume removed on each plasmapheresis during the LVPE period was between 650 mL and 880 mL (depending on the patient body weight). After the LVPE, albumin 20% was infused depending on treatment arm randomization (LA or HA) approximately 20 g to 40 g albumin. Further, the HA+F treatment arm and one LA+F arm received infusions of IVIG (F) as indicated above.

For the purpose of the analysis, two comparison groups were considered: control/placebo patients and the pooled PE-Alb-treated patients (the three treated groups).

### Sampling

The AMBAR study recruited patients (and, therefore, collected serum and CSF samples) from 2012 to 2017. In this study only samples stored for up to 4 years since collection until analysis (i.e., 2016 onwards) were selected and analyzed.

Serum samples were taken at 8 visits across the study (in 3 of them, immediately before and after PE-Alb) being in total 11 time points: baseline; TPE 1 (week 1: pre- and post-TPE); TPE 6 (week 6; pre-TPE); intermediate visit (week 7-8); LVPE 1 (month 3: pre- and post-LVPE); LVPE 7 (month 9: pre- and post-LVPE); LVPE 12 (month 14: pre-LVPE); and final visit (month 14) . . . .

That represented a total of 1674 samples for analysis, collected from 142 patients: 1312 serum (8 visits).

### MIP-1α Biomarker Assays

Three electrochemiluminescence MSD V-PLEX Panel kits (Meso Scale Diagnostic LLC; Rockville MD; USA) were used for biomarker analysis in serum and CSF.

As a quality analysis criteria, biomarker was considered as evaluable if it was detectable in more than 30% (ad-hoc value) of the samples at both baseline and final visit.

### Data Analysis and Statistics

The baseline distribution of MIP-1a levels in the control and the treated groups were compared by means of a Wilcoxon test.

In relation to treatment effects evaluation, two different kind of analysis have been performed:
a) Lasting effects: effects of PE-Alb treatment over time; and
b) Acute effects; effects of PE-Alb treatment associated to each plasma exchange procedure.

Lasting effects were analyzed over time in pooled PE-Alb treated patients compared to controls as the levels change from baseline through a Mixed Model for Repeated Measures (MMRM). For this analysis, samples post PE were not considered. Fixed effects factors for month, treatment group, and month*treatment interaction, with adjustment for age, AD severity (baseline MMSE score) and baseline biomarker levels were used, and patient was included as a repeated factor in the model.

BM=α+β1 Group*Visit (time)+β2 MMSE0+β3 Age+β4 BM0+(1|Patient)

BM: biomarker level; BM0: biomarker level at baseline; Group: PE-Alb-treated or placebo; Visit: baseline, pre-FPE1, pre-FPE6, Intermediate, pre-LVPE1, pre-LVPE7, pre-LVPE9, pre-LVPE12 or Final; MMSE0: baseline MMSE score.

Reported p-values were adjusted by Benjamini-Hochberg procedure to account for multiple comparisons and decrease the False Discovery Rate (FDR). An adjusted p-value of 0.05 was used as statistical significance threshold. Least Square Means (LSM) (±standard error of the mean [SEM]) was used to plot the differences versus the baseline, and the ratio between groups (pooled PE-Alb-treated versus placebo) of the LSM was calculated per each timepoint. All measures were log 2 normalized and standardized prior to analyses so that β1-coefficients of the interaction month*treatment can be interpreted as standardized effect size.

Acute effects, being changes in MIP-1a levels directly related to the single PE-Alb procedure, were assessed with samples collected immediately before and after PE-Alb (TPE 1 and LVPE 7). Differences between the two visits were represented as effect size, calculated as the rank biserial correlation for non-parametric test.

### Clinical Assessments

The following clinical and neuropsychological measurements were performed: Alzheimer's Disease Cooperative Study-Activities of Daily Living (ADCS-ADL) as a functional scale; Alzheimer's Disease Assessment Scale-Cognitive Subscale (ADAS-Cog) as a cognitive scale; and two global assessment of change scales-Clinical Dementia Rating Sum of Boxes (CDR-sb), and Alzheimer's Disease Cooperative Study-Clinical Global Impression of Change (ADCS-CGIC).

There were four visits in which both biomarker levels and clinical outcome value were available: baseline, intermediate visit (at the end of TPE), LVPE 7, and final visit.

The ADAS-Cog is an instrument specifically designed to evaluate the severity of the fundamental alterations in cognitive and behavioral function that are characteristic of patients with AD.

Functional ability is assessed by means of the ADCS-ADL test, which offers detailed descriptions of each activity and requests the informer to describe the actions or behaviors observed.

CDR-Sb, a clinical test validated in patients with AD that assesses six domains: memory, orientation, judgment and problem solving, social and occupational activities, domestic activities and hobbies, and personal care; and ADCS-CGIC, an instrument for the reliable assessment of global change from the baseline in a clinical trial.

Importantly, the evaluators/raters of the tests in the trial have no access to any information allowing them to identify patient assignment to treatment. Blinding of the evaluators to patient treatment is confirmed when evaluators sign a document to that effect.

The possible relationship between changes in the levels of serum inflammatory markers across the study, and the clinical endpoints was investigated through a repeated measures correlation analysis as well as through a parsimonious and an extended MMRM which differ on the amount of included variables. The extended model includes all the variables used on the MMRM for the lasting effects analysis previously described.

### Parsimonious Model:

CFBclinical endpoint=α+β1·BM*Group*Visit(time)+(1|Patient)

### Extended Model:

CFBclinical endpoint=β1·BM*Group*Visit(time)+β2 MMSE0+β3 Age+β4 Clinical_Endpoint0+(1|Patient)

CFB: change from baseline value; BM: biomarker level; Group: PE-Alb-treated or placebo; Visit: Baseline, IV, pre-LVPE7 or Final; MMSE0: baseline MMSE score; Clinical_Endpoint₀: clinical endpoint value at baseline.

In order to ensure that the statistical differences found between MIP1α biomarker and the clinical outcomes were not derived or influenced by other variables a mediation analysis was performed. Study demographic information and laboratory variables available were added to the previous parsimonious model, to test its influence on the statistical significance and on the B 1 estimate (BM*Group*Visit (time) interaction

All statistical analyses were performed with R version 4.1 (https://cran.r-project.org/).

### Results

### Baseline Levels: Placebo Vs Treated

At baseline, there were no statistically significant differences between control and PE-Alb-treated samples (pooled) of serum.

### Lasting Effect

In serum, MIP1α showed LSM ratio >1 (i.e., ratio favors of PE-Alb treated groups) in all timepoints including the end of the intensive TPE period (month 2). Further, MIP1α showed a LSM ratio >1 at the end of the maintenance LVPE period (month 14) (Table 1).

Statistically significant differences (p<0.05; uncorrected) from baseline in at least one time point (0.2, 1.5, 2, 2.2, 9, 13.5, and 14 months) was observed. After FDR correction, differences remained significant in MIP-1α at final visit.

TABLE 1
LSM ratio of Mip1a;

| Month | Ratio | p-val |
|---|---|---|
| 0.2 | 1.071 | 0.318 |
| 1.5 | 1.174 | 0.020 |
| 2 | 1.158 | 0.031 |
| 2.2 | 1.010 | 0.879 |
| 9 | 1.028 | 0.697 |
| 13 | 1.072 | 0.301 |
| 14 | 1.270 | 0.001^{#} |

^{#}indicates adjusted p-value < 0.05 (BH).

### Acute Effects

Levels in the placebo samples remained unchanged when comparing before and after sham PE-Alb in the two procedures assessed: TPE 1 and LVPE 7.

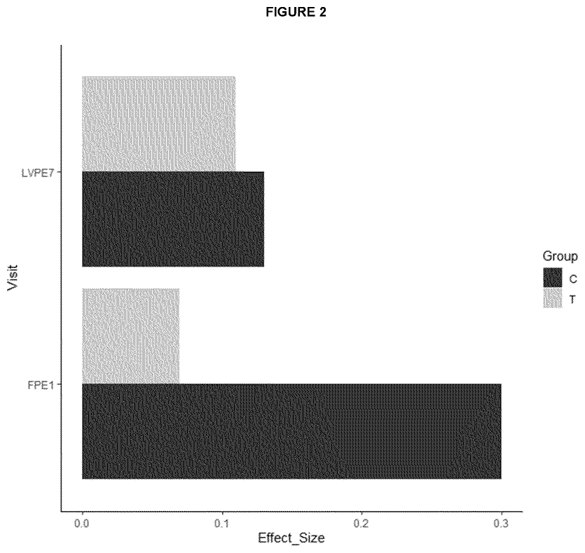

Conversely, in PE-Alb-treated samples, MIP1α biomarker levels significantly decreased (negative fold-change; p<0.05) after TPE 1 and LVPE 7 (FIG. 2).

### Correlation Studies

Results obtained from repeated measures correlations show that control patients have a positive correlation (r values between 0.33 and 0.23) between Mip1α levels and three of the clinical outcomes analyzed (ADAS-Cog, CDR-SB and ADCS-CGIC), being significant in ADAS-Cog and CDR-SB. Conversely, in treated patients this positive correlation between Mip1α and clinical endpoints is lost (Table 2).

TABLE 2
Repeated measures correlations between Mip1α levels
and clinical outcomes in Control and Treated patients.

| | ADAS-Cog | | ADCS-ADL | | CDR-SB | | ADCS-CGIC | |
|---|---|---|---|---|---|---|---|---|
| | r | p-val | r | p-val | r | p-val | r | p-val |
| Control | 0.328 | 0.002 | −0.175 | 0.102 | 0.293 | 0.006 | 0.223 | 0.096 |
| Treated | −0.081 | 0.267 | 0.058 | 0.433 | −0.061 | 0.519 | −0.052 | 0.476 |

MMRM results show that Mip1α levels in treated patients along time significantly correlated (p<0.05) with the change from baseline in three out of the four neuropsychological tests assessed during the study (ADAS-Cog, CDR-sb, and ADCS-CGIC). Significance results coincided using both the parsimonious and extended MMRM models. Beta and p values and neuropsychological tests are shown in Table 3 below.

TABLE 3
Estimates from the triple interaction (β 1 · Mip1α * Group * Visit (time)).

| | ADAS-Cog | | CDR-SB | | ADCS-Cgic | |
|---|---|---|---|---|---|---|
| | Estimates | p-value | Estimates | p-value | Estimates | p-value |
| CTL vs Treated | −0.73 | 0.021 | −0.24 | 0.021 | −0.16 | 0.011 |
| (Parsimonius model) | | | | | | |
| CTL vs Treated | −0.69 | 0.028 | −0.24 | 0.022 | −0.15 | 0.014 |
| (Extended model) | | | | | | |

Thus, the results show that there is a clear relationship between MIP-1a marker and clinical outcome. Statistical significance and estimates of the triple interaction term of the parsimonious model were maintained when evaluating possible mediated effects of demographic and laboratory variables recollected in the change from baseline of the neuropsychological test assessed.

### Predicted Trajectories

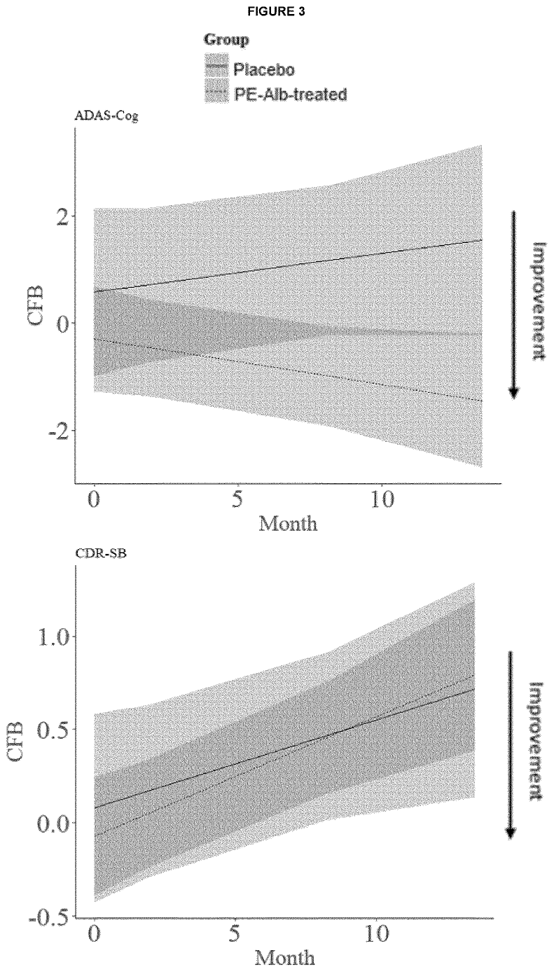

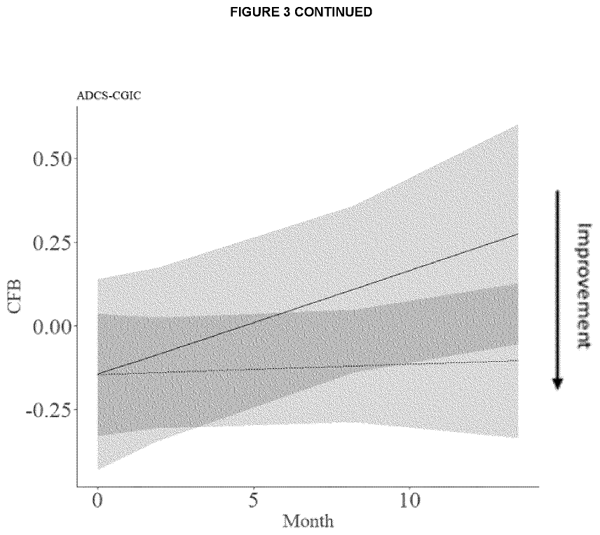

Predicted trajectories of clinical outcomes (change from baseline at the end of the study) based on levels of MIP-1a are shown in FIG. 3. ADAS-Cog score showed a longitudinal improvement in PE-Alb-treated patients. CDR-sb showed no clear differences between placebo and PE-Alb-treated patients. ADCS-CGIC trajectories for placebo group showed worsening over time, while PE-Alb-treated patients remained stable.

### DISCUSSION

Acute effects showed that levels of MIP1α decreased after PE-Alb, in both TPE and LVPE modalities. Generally, a larger fold-change and effect size was observed after TPE than after LVPE in treated patients.

Lasting effects studies evidenced that PE-Alb induced changes in patients' serum MIP1α profile across the study.

Although not with a fully consistent homogeneity across time points, a reduction of the inflammation status was overall observed in treated patients versus placebo, which was particularly evidenced at month 2, the end of intensive period and at month 14, the end of study (FDR corrected significant increase decrease in MIP1α in serum).

MIP-α is a major factor produced by macrophages and monocytes that is crucial for immune responses towards infection and inflammation. MIP1α is also produced by neurons and microglia. Results of a recent study indicated that inflammatory plasma molecules were not increased but reduced in moderate-stage AD patients [Koca S. et al 2022; Neurosci Lett; 786:136799].

The correlation results observed in repeated measurements correlation analysis and MMRM suggest serum MIP1α as an indicator of treatment efficacy or effectiveness for AD. Changes in MIP1α levels significantly correlated with those of three neuropsychological tests: ADAS-Cog, CDR-sb, and ADCS-CGIC and thus alongside the positive outcome of clinical endpoints.

1. An ex vivo method for assessing the effectiveness or efficacy of a treatment in a subject suffering from a neurodegenerative disease, said method comprising:
a) determining the expression level of a MIP1α expression product in a biological sample from said subject;
b) repeating step a) using a biological sample obtained from said subject after a period of treatment; and
c) comparing the expression level in b) with that in a) to assess the effectiveness or efficacy of the treatment;
wherein a decrease or a no increase in the expression level in b) compared to a) indicates that the treatment is effective or efficacious in said subject and/or that said subject is more likely to exhibit a favorable clinical response to said treatment; and wherein an increased expression level in b) compared to a) is indicative of a decreased likelihood of favorable clinical response to said treatment.

2. Method according to claim 1, wherein said neurodegenerative disease is selected from the group consisting of: Alzheimer's disease, Parkinson's disease, synucleinopathies, Mild Cognitive Impairment, Diffuse Lewy body disease, Dementia with Lewy bodies type, amyotrophic lateral sclerosis, Pick's disease, tauopathies, trinucleotide repeat expansion diseases such as (without limitation) Huntington's disease and spinocerebellar ataxias, Creutzfeldt-Jakob disease, frontotemporal dementia, and combinations thereof.

3. Method according to claim 2, wherein said neurodegenerative disease is Alzheimer's disease.

4. Method according to claim 3, wherein said Alzheimer's disease is mild or moderate Alzheimer's Disease.

5. Method according to claim any one of claims 1 to 4, wherein said treatment comprises plasma exchange treatment.

6. Method according to claim 5, wherein said plasma exchange treatment comprises therapeutic plasma exchange (TPE).

7. Method according to claim 5 or 6, wherein said plasma exchange treatment comprises low volume plasma exchange (LVPE).

8. Method according to any one of claims 5 to 7, wherein said plasma exchange treatment comprises a combination of therapeutic plasma exchange (TPE) and low volume plasma exchange (LVPE).

9. Method according to anyone of claims 5 to 8, wherein said plasma exchange treatment includes albumin replacement.

10. Method according to claim 9, wherein said albumin is administered at an amount of between 10 g and 60 g of albumin per Litre of plasma removed.

11. Method according to any one of claims 5 to 10, wherein said plasma exchange treatment additionally includes administering immunoglobulin.

12. Method according to any one of claims 1 to 11, wherein said biological sample is selected from the group consisting of a blood sample, plasma sample, serum sample and a cerebrospinal fluid sample.

13. Method according to any one of claims 1 to 12, wherein the treatment referred to in step b) is therapeutic plasma exchange (TPE) treatment.

14. Method according to any one of claims 1 to 13, wherein the treatment referred to in step b) is low volume plasma exchange (LVPE) treatment.

15. Method according to any one of claims 1 to 14, wherein said MIP1α expression product is a MIP1α RNA transcript or a MIP1α protein.

16. Use of MIP1α as a biomarker of plasma exchange clinical treatment effectiveness or efficacy in a patient suffering from a neurodegenerative disease.

17. A kit for predicting whether a patient diagnosed with a neurodegenerative disease, disorder or condition is likely to be responsive or non-responsive to a plasma exchange treatment, or for assessing the effectiveness or efficacy of plasma exchange in treating a patient diagnosed with a neurodegenerative disease, disorder or condition, said kit comprising reagents useful for determining the patients level of MIP1α.

18. The kit of claim 17, comprising an antibody, or antigen-binding fragment thereof, that specifically binds to MIP1α.
